(19) United States
(12) Patent Application Publication
(10) Pub. No.: US 2024/0307968 A1
(43) Pub. Date: Sep. 19, 2024

(54) SINTERING STATE COMBINATIONS

(71) Applicant: HEWLETT-PACKARD DEVELPOMENT COMPANY, L.P., Spring, TX (US)

(72) Inventors: Zi-Jiang YANG, Shanghai (CN); Chuang GAN, Shanghai (CN); Lei CHEN, Shanghai (CN); Carlos Alberto LOPEZ COLLIER DE LA MARLIERE, Guadalajiara, JAL (MX); Yu XU, Shanghai (CN); Juheon LEE, Palo Alto, CA (US); Jun ZENG, Palo Alto, CA (US)

(73) Assignee: HEWLETT-PACKARD DEVELPOMENT COMPANY, L.P., Spring, TX (US)

(21) Appl. No.: 18/578,265

(22) PCT Filed: Jul. 14, 2021
(86) PCT No.: PCT/US2021/041558

§ 371 Date: Jan. 10, 2024

(51) Int. Cl. B22F 10/80; B33Y 50/00

(52) U.S. Cl. CPC B22F 10/80; B33Y 50/00

(57) ABSTRACT

Examples of methods are described herein. In some examples, a method includes predicting a first sintering state of an object using a first machine learning model trained based on a first time segment. In some examples, the method includes predicting a second sintering state of the object using a second machine learning model trained based on a second time segment. In some examples, the method includes combining, using a fusion machine learning model, the first sintering state and the second sintering state to produce an overall sintering state.

### BACKGROUND

Three-dimensional (3D) solid parts may be produced from a digital model using additive manufacturing. Additive manufacturing may be used in rapid prototyping, mold generation, mold master generation, and short-run manufacturing. Additive manufacturing involves the application of successive layers of build material. This is unlike some machining processes that often remove material to create the final part. In some additive manufacturing techniques, the build material may be cured or fused.

### BRIEF DESCRIPTION OF THE DRAWINGS

FIG. 1 is a flow diagram illustrating an example of a method for determining a sintering state combination;

FIG. 2 is a diagram illustrating an example of a plot of displacement and temperature in accordance with some of the techniques described herein;

FIG. 3 is a block diagram of an example of an apparatus that may be used in combining sintering states;

FIG. 4 is a block diagram illustrating an example of a computer-readable medium for combining sintering states;

FIG. 5 is a diagram illustrating an example of a machine learning model architecture that may be utilized in accordance with some of the techniques described herein; and

FIG. 6 is a block diagram illustrating an example of components that may be utilized in accordance with some examples of the techniques described herein.

### DETAILED DESCRIPTION

Additive manufacturing may be used to manufacture three-dimensional (3D) objects. 3D printing is an example of additive manufacturing. Metal printing (e.g., metal binding printing, binder jet, Metal Jet Fusion, etc.) is an example of 3D printing. In some examples, metal powder may be glued at certain voxels. A voxel is a representation of a location in a 3D space (e.g., a component of a 3D space). For instance, a voxel may represent a volume that is a subset of the 3D space. In some examples, voxels may be arranged on a 3D grid. For instance, a voxel may be cuboid or rectangular prismatic in shape. In some examples, voxels in the 3D space may be uniformly sized or non-uniformly sized. Examples of a voxel size dimension may include 25.4 millimeters (mm)/150˜170 microns for 150 dots per inch (dpi), 490 microns for 50 dpi, 2 mm, 4 mm, etc. The term “voxel level” and variations thereof may refer to a resolution, scale, or density corresponding to voxel size.

Some examples of the techniques described herein may be utilized for various examples of additive manufacturing. For instance, some examples may be utilized for metal printing. Some metal printing techniques may be powder-based and driven by powder gluing and/or sintering. Some examples of the approaches described herein may be applied to area-based powder bed metal printing, such as binder jet, Metal Jet Fusion, and/or metal binding printing, etc. Some examples of the approaches described herein may be applied to additive manufacturing where an agent or agents (e.g., latex) carried by droplets are utilized for voxel-level powder binding.

In some examples, metal printing may include two phases. In a first phase, the printer (e.g., print head, carriage, agent dispenser, and/or nozzle, etc.) may apply an agent or agents (e.g., binding agent, glue, latex, etc.) to loose metal powder layer-by-layer to produce a glued precursor (or “green”) object. A precursor object is a mass of metal powder and adhesive. In a second phase, a precursor object may be sintered (e.g., heated) to produce an end object. For example, the glued precursor object may be placed in a furnace or oven to be sintered to produce the end object. Sintering may cause the metal powder to fuse, and/or may cause the agent to be burned off. An end object is an object formed from a manufacturing procedure or procedures. In some examples, an end object may undergo a further manufacturing procedure or procedures (e.g., support removal, polishing, assembly, painting, finishing, etc.). A precursor object may have an approximate shape of an end object.

The two phases of some examples of metal printing may present challenges in controlling the shape (e.g., geometry) of the end object. For example, the application (e.g., injection) of agent(s) (e.g., glue, latex, etc.) may lead to porosity in the precursor part, which may significantly influence the shape of the end object. In some examples, metal powder fusion (e.g., fusion of metal particles) may be separated from a layer-by-layer printing procedure, which may limit control over sintering and/or fusion.

In some examples, metal sintering may be performed in approaches for metal injection molded (MIM) objects and/or binder jet (e.g., MetJet). In some cases, metal sintering may introduce a deformation and/or change in an object varying from 25% to 50% depending on precursor object porosity. A factor or factors causing the deformation (e.g., visco-plasticity, sintering pressure, yield surface parameters, yield stress, and/or gravitational sag, etc.) may be captured and applied for shape deformation simulation. Some approaches for metal sintering simulation may provide science-driven simulation based on first principle sintering physics and/or empirical experiment-adjusted (e.g., over-specific) thermal profile determination. For instance, factors including thermal profile and/or yield curve may be utilized to simulate object deformation due to shrinkage and/or sagging, etc. In some approaches, metal sintering simulation may provide science driven prediction of an object deformation and/or compensation for the deformation. Some simulation approaches may provide relatively high accuracy results at a voxel level for a variety of geometries (e.g., from less to more complex geometries). Due to computational complexity, some examples of physics-based simulation engines may take a relatively long period to complete a simulation. For instance, simulating transient and dynamic sintering of an object may take from tens of minutes to several hours depending on object size. In some examples, larger object sizes may increase simulation runtime. For example, a 12.5 centimeter (cm) object may take 218.4 minutes to complete a simulation run. Some examples of physics-based simulation engines may utilize relatively small increments (e.g., time periods) in simulation to manage the nonlinearity that arises from the sintering physics. Accordingly, it may be helpful to reduce simulation time.

Some examples of the techniques described herein may utilize a machine learning model or models. Machine learning is a technique where a machine learning model is trained to perform a task or tasks based on a set of examples (e.g., data). Training a machine learning model may include determining weights corresponding to structures of the machine learning model. Artificial neural networks are a kind of machine learning model that are structured with nodes, model layers, and/or connections. Deep learning is a kind of machine learning that utilizes multiple layers. A deep neural network is a neural network that utilizes deep learning.

Examples of neural networks include convolutional neural networks (CNNs) (e.g., basic CNN, deconvolutional neural network, inception module, residual neural network, etc.), recurrent neural networks (RNNs) (e.g., basic RNN, multi-layer RNN, bi-directional RNN, fused RNN, clockwork RNN, etc.), graph neural networks (GNNs), etc. Different depths of a neural network or neural networks may be utilized in accordance with some examples of the techniques described herein.

In some examples of the techniques described herein, a deep neural network may predict or infer a sintering state. A sintering state is data representing a state of an object in a sintering procedure. For instance, a sintering state may indicate a characteristic or characteristics of the object at a time during the sintering procedure. In some examples, a sintering state may indicate a physical value or values associated with a voxel or voxels of an object. Examples of a characteristic(s) that may be indicated by a sintering state may include displacement, porosity, a displacement rate of change, and/or velocity, etc. Displacement is an amount of movement (e.g., distance) for all or a portion (e.g., voxel(s)) of an object. For instance, displacement may indicate an amount and/or direction that a part of an object has moved during sintering over a time period (e.g., since beginning a sintering procedure). Displacement may be expressed as a displacement vector or vectors at a voxel level. Porosity is a proportion of empty volume or unoccupied volume for all or a portion (e.g., voxel(s)) of an object. A displacement rate of change is a rate of change (e.g., velocity) of displacement for all or a portion (e.g., voxel(s)) of an object.

In some examples, simulating and/or predicting sintering states may be performed in a voxel space. A voxel space is a plurality of voxels. In some examples, a voxel space may represent a build volume and/or a sintering volume. A build volume is a 3D space for object manufacturing. For example, a build volume may represent a cuboid space in which an apparatus (e.g., computer, 3D printer, etc.) may deposit material (e.g., metal powder, metal particles, etc.) and agent(s) (e.g., glue, latex, etc.) to manufacture an object (e.g., precursor object). In some examples, an apparatus may progressively fill a build volume layer-by-layer with material and agent during manufacturing. A sintering volume may represent a 3D space for object sintering (e.g., oven). For instance, a precursor object may be placed in a sintering volume for sintering. In some examples, a voxel space may be expressed in coordinates. For example, locations in a voxel space may be expressed in three coordinates: x (e.g., width), y (e.g., length), and z (e.g., height).

In some examples, a sintering state may indicate a displacement in a voxel space. For instance, a sintering state may indicate a displacement (e.g., displacement vector(s), displacement field(s), etc.) in voxel units and/or coordinates. In some examples, a sintering state may indicate a position of a point or points of the object at a second time, where the point or points of the object at the second time correspond to a point or points of the object at the first time (and/or at a time previous to the first time). A displacement vector may indicate a distance and/or direction of movement of a point of the object over time. For instance, a displacement vector may be determined as a difference (e.g., subtraction) between positions of a point over time (in a voxel space, for instance).

In some examples, a sintering state may indicate a displacement rate of change (e.g., displacement “velocity”). For instance, a machine learning model may produce a sintering state that indicates the rate of change of the displacements. For example, a machine learning model (e.g., deep learning model for inferencing) may predict a displacement velocity for an increment (e.g., prediction increment).

In some cases, challenges may arise in deploying a single neural network to predict sintering states over an entire sintering procedure. For example, sintering deformation may result from a non-linear combination of multiple dynamic effects, such as elastic strain, plastic strain, and/or viscous strain. In some examples, sintering temperature may vary over the sintering procedure. To predict deformation over a sintering procedure (e.g., time increments of a sintering procedure), a machine learning model (e.g., deep learning model) may attempt to learn a complex function of the temperature profile.

To manage the sintering physics complexities over time, it may be helpful to train multiple machine learning models, where each machine learning model corresponds to a different sintering stage of the sintering procedure. A sintering stage is a period during a sintering procedure. For example, a sintering procedure may include multiple sintering stages (e.g., 2, 3, 4, etc., sintering stages). In some examples, each sintering stage may correspond to different circumstances (e.g., different temperatures, different heating patterns, different temperature raise rates, different temperature heating stages, different periods during the sintering procedure, etc.). For instance, sintering dynamics at different temperatures and/or sintering stages may have different deformation rates. Multiple machine learning models (e.g., deep learning models) may be trained to be tailored to different sintering stages. In some examples, each machine learning model may produce a prediction. The predictions may be combined, fused, and/or synthesized to produce a final prediction. In some examples, different machine learning models (e.g., GNNs) may be used to learn the deformation dynamics of different stages to produce multiple predictions. Sintering may be a complex physical process, with various factors and forces involved. In some examples, sintering may include a dominant or combined forces for different sintering stages. In some examples, different machine learning models may be utilized to learn specific deformation dynamics of different stages to provide faster learning and/or more accurate results.

In some examples, another machine learning model (e.g., an RNN) may be used to fuse the predictions to produce an overall prediction. Since the dynamics for each individual sintering stage are simpler than all of the dynamics over the entire sintering procedure, the size of each machine learning model may be smaller than a size of a monolithic machine learning model. Smaller machine learning models may be more efficient to train. In some examples, multiple trained machine learning models for multiple stages and fusing may lead to results with increased accuracy and/or larger automation coverage. For example, specific sintering dynamics of a “gap” or “transitioning” in a sintering procedure (which may not be covered entirely by a trained sintering stage machine learning model in some examples), may be learned by a fusion machine learning model from training data. For instance, a fusion machine learning model may learn how to fuse results from multiple machine learning models and may learn to accurately predict an overall result.

A time period in a sintering procedure may be referred to as an increment or time increment. A time period spanned in a prediction (by a machine learning model or models, for instance) may be referred to as a prediction increment. For example, a deep neural network may infer a sintering state at second time based on a sintering state (e.g., displacement) at a first time, where the second time is subsequent to the first time. A time period spanned in simulation may be referred to as a simulation increment. In some examples, a prediction increment may be different from (e.g., greater than) the simulation increment. In some examples, a prediction increment may be an integer multiple of a simulation increment. For instance, a prediction increment may span and/or replace many simulation increments.

In some examples, a prediction of a sintering state at a second time may be based on a simulated sintering state at a first time. For instance, a simulated sintering state at the first time may be utilized as input to a machine learning model to predict a sintering state at the second time. Predicting a sintering state using a machine learning model may be performed more quickly than simulating a sintering state. For example, predicting a sintering state at the second time may be performed faster than determining the sintering state at the second time through simulation. For instance, a relatively large number of simulation increments may be utilized, and each simulation increment may take a quantity of time to complete. For instance, a simulation may advance in simulation increments of dt. A machine learning model (e.g., neural network) may produce a prediction covering multiple simulation increments (e.g., 10*dt, 100*dt, etc.). Utilizing prediction (e.g., machine learning, inferencing, etc.) to replace some simulation increments may enable determining a sintering state in less time (e.g., more quickly). For example, utilizing machine learning (e.g., a deep learning inferencing engine) in conjunction with simulation may allow larger (e.g., ×10) increments (e.g., prediction increments) to increase processing speed while preserving accuracy.

Some examples of the techniques described herein may be performed in an offline loop. An offline loop is a procedure that is performed independent of (e.g., before) manufacturing, without manufacturing the object, and/or without measuring (e.g., scanning) the manufactured object.

Throughout the drawings, identical reference numbers may or may not designate similar or identical elements. Similar numbers may or may not indicate similar elements. When an element is referred to without a reference number, this may refer to the element generally, with or without limitation to any particular drawing or figure. The drawings may or may not be to scale, and the size of some parts may be exaggerated to more clearly illustrate the example shown. Moreover, the drawings provide examples in accordance with the description. However, the description is not limited to the examples provided in the drawings.

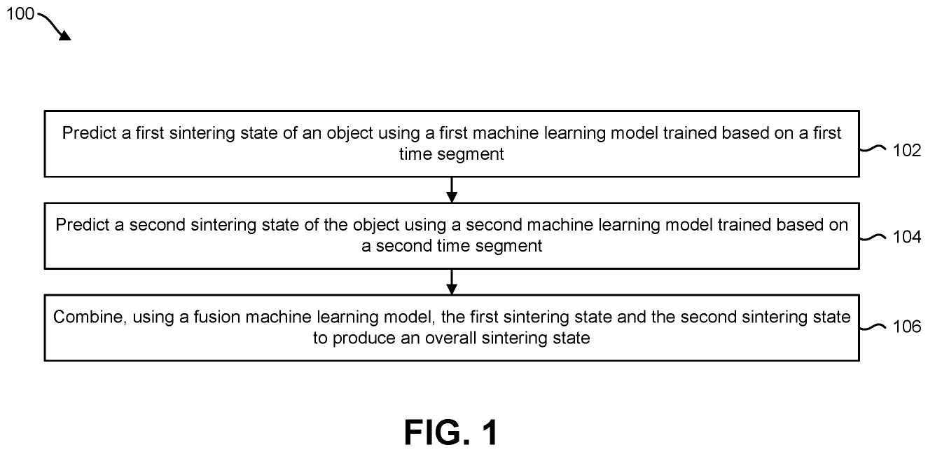

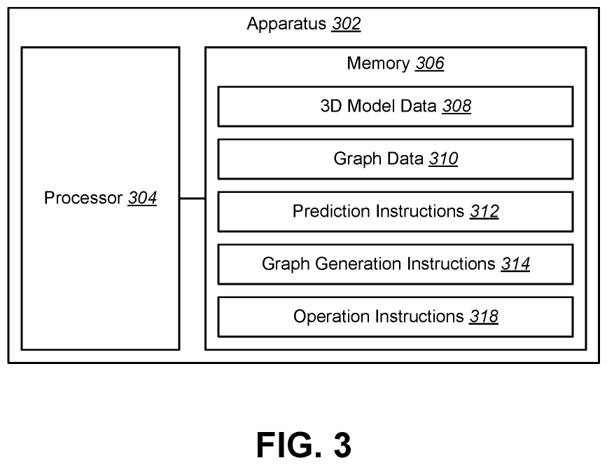

FIG. 1 is a flow diagram illustrating an example of a method 100 for determining a sintering state combination. The method 100 and/or an element or elements of the method 100 may be performed by an apparatus (e.g., electronic device). For example, the method 100 may be performed by the apparatus 302 described in relation to FIG. 3.

The apparatus may predict 102 a first sintering state of an object using a first machine learning model trained based on a first time segment (and/or based on a first sintering stage). The object may be represented by an object model. An object model is a geometrical model of an object. For instance, an object model may be a three-dimensional (3D) model representing an object. Examples of object models include computer-aided design (CAD) models, mesh models, 3D surfaces, etc. An object model may be expressed as a set of points, surfaces, faces, vertices, etc. In some examples, the apparatus may receive an object model from another device (e.g., linked device, networked device, removable storage, etc.) or may generate the 3D object model.

A time segment is a duration of time. For example, a time segment may correspond to a span of time for a sintering procedure. In some examples, a time segment may be included in a sintering stage. For instance, the first time segment may be within a first sintering stage and/or may be associated with a first sintering stage. In some examples, the first machine learning model may be trained based on simulated sintering states from the first time segment. In some examples, each time segment and/or sintering stage may be separated and/or determined based on a physical insight (that a heating pattern affects the deformation profile, for instance).

In some examples, the first machine learning model is a first GNN. A GNN is a neural network that operates on graph data. Graph data is data indicating a structure of nodes and edges. In some examples, the graph data may represent the object. For instance, nodes of the graph data may correspond to voxels of the object and/or may represent voxels of the object. In some examples, edges of the graph data may represent interactions between nodes (e.g., voxel-to-voxel interactions). In some examples, the graph data may indicate a graph (e.g., nodes and edges) at a time (e.g., increment) of a sintering procedure. In some examples, a graph may include another factor or factors. For instance, a graph may include a global temperature at a time (e.g., increment) of the sintering procedure. In some examples, each node may include an attribute or attributes. For instance, a node may indicate a velocity and/or displacement of a voxel. For example, a node may include a vector indicating velocity in three dimensions (e.g., x, y, z). For instance, a node may indicate a displacement velocity for a voxel. In some examples, a node may include a series of vectors for a quantity of increments (e.g., velocities for the last three increments). In some examples, a sintering state may be expressed as a graph or graph data. For instance, the first machine learning model may predict the first sintering state (e.g., a graph) based on a previous sintering state or states (e.g., a graph indicating a sintering state at a previous increment).

The apparatus may predict 104 a second sintering state of the object using a second machine learning model trained based on a second time segment. For instance, the second time segment may be within a second sintering stage and/or may be associated with a second sintering stage. The second machine learning model may be trained based on simulated sintering stages from the second time segment.

In some examples, the second machine learning model is a second GNN. For instance, the second machine learning model may predict the second sintering state (e.g., a graph) based on a previous sintering state (e.g., a graph indicating a sintering state at a previous increment).

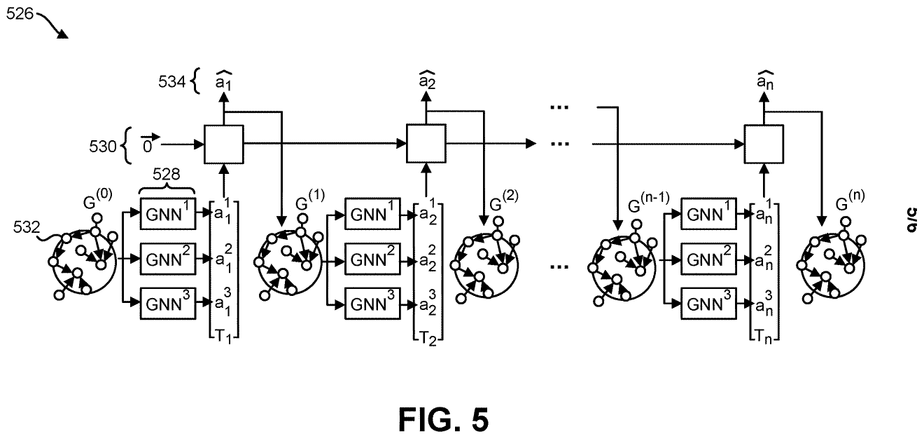

In some examples, the machine learning models described herein may be trained using training data from a training simulation or simulations. For example, the machine learning model may utilize training sintering states (e.g., displacement, displacement rate of change, velocity, etc., corresponding to simulation increment(s)) from a sintering stage as input and as ground truth during training. Examples of machine learning model architectures that may be utilized in accordance with the techniques described herein are given in relation to FIG. 5. For instance, the first machine learning model and the second machine learning model may be neural network(s), GNN(s), etc.

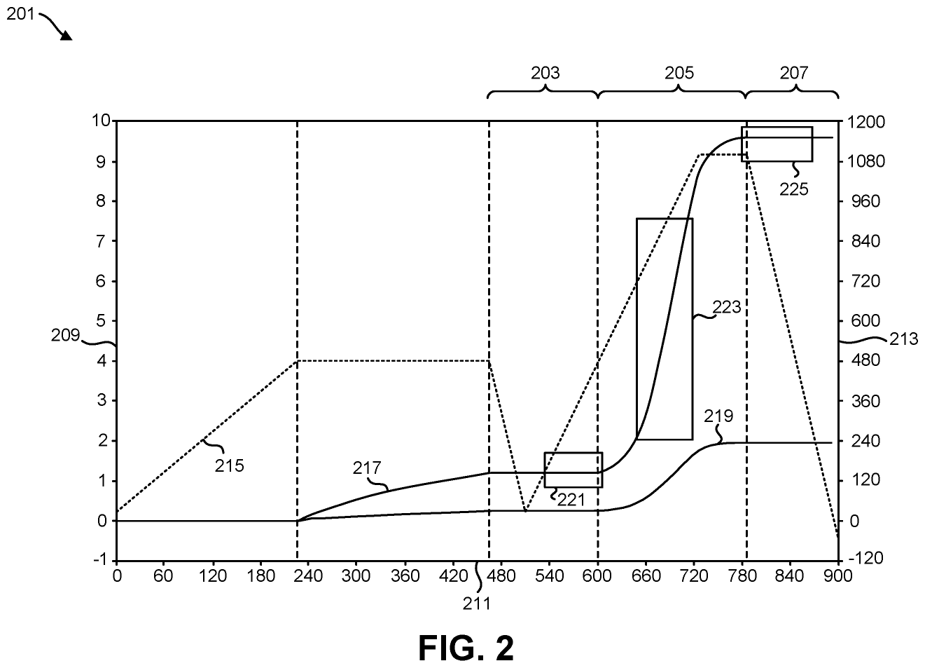

In some examples, the first machine learning model and the second machine learning model may be trained using corresponding sintering stage data. For instance, the respective machine learning models may be trained with different training data. For example, the first machine learning model may be trained with data from a first time segment of a first stage of a training simulation and a second machine learning model may be trained with data from a second time segment of a second stage of the training simulation (and/or another training simulation). In some examples, machine learning models corresponding to different sintering stages may have similar or the same architectures and/or may be trained with different training data. While a first machine learning model corresponding to a first sintering stage and a second machine learning model corresponding to a second sintering stage are given as examples in relation to FIG. 1, some examples of the techniques described herein may utilize more (e.g., 3, 4, 5, etc.) machine learning models trained corresponding to more (e.g., 3, 4, 5, etc.) sintering stages. For instance, a machine learning model(s) may be utilized corresponding to a sintering stage(s) based on a heating pattern, thermal profile, and/or other factor(s). An example of sintering stages is given in relation to FIG. 2.

In some examples, the machine learning models may have a fixed prediction increment at a time (e.g., a prediction increment at time TA to time TB) when deployed. A fixed prediction increment may be useful for defined sintering temperature schedules.

The apparatus may combine 106, using a fusion machine learning model, the first sintering state and the second sintering state to produce an overall sintering state. For example, the fusion machine learning model may be trained to combine (e.g., fuse) sintering states to produce an overall sintering state that matches a simulated sintering state (e.g., ground truth). In some examples, the fusion machine learning model is an RNN.

In some examples, the fusion machine learning model is trained to learn a dynamic of a sintering procedure based on a first time segment (e.g., the first time segment of the first sintering stage) and a second time segment (e.g., the second time segment of the second sintering stage). More or fewer time segments may be utilized in accordance with some examples of the techniques described herein. For instance, the fusion machine learning model (e.g., an RNN) may utilize a simulated sintering state (e.g., ground truth, simulated overall sintering state) and outputs (e.g., predicted sintering states) of machine learning models (e.g., GNNs) to learn a dynamic or dynamics of a sintering procedure. For example, the fusion machine learning model may learn dynamics of different time segments (e.g., sintering stages), such as elastic strain, plastic strain, and/or viscous strain. In some examples, the fusion machine learning model may learn dynamics of transitioning between time segments (e.g., sintering stages). While it may be difficult to quantitatively formulate specific physical effects of transitions of a sintering procedure, the fusion machine learning model may learn dynamics of transitioning. In some examples, the training of the fusion machine learning model may be performed using input data such as sintering temperature increments and machine learning model (e.g., GNN) predictions, without correction using numeric method analysis.

It may be helpful to fuse machine learning model (e.g., GNNs') predictions using the fusion machine learning model. For instance, fusing the prediction may enable capturing the history of temperature changes, thereby smoothly switching the dynamics of predictions between different sintering stages.

In some examples, the first machine learning model, the second machine learning model, and the fusion machine learning model may produce the overall sintering state based on a quantity of initial simulated sintering states. In some examples, the apparatus may voxelize the object (e.g., 3D object model) to produce voxels of the object. For instance, the apparatus may convert the 3D object model into voxels representing the object. The voxels may represent portions (e.g., rectangular prismatic subsets and/or cubical subsets) of the object in 3D space.

In some examples, the apparatus may simulate sintering of the voxels to produce a quantity of initial simulated sintering states. For example, the apparatus may utilize a physics engine to produce a quantity of initial simulated sintering states. A physics engine is hardware (e.g., circuitry) or a combination of instructions and hardware (e.g., a processor with instructions) to simulate a physical phenomenon or phenomena. In some examples, the physics engine may simulate material (e.g., metal) sintering. For example, the physics engine may simulate physical phenomena on an object (e.g., object model) over time (e.g., during sintering). The simulation may indicate deformation effects (e.g., shrinkage, sagging, etc.). In some examples, the physics engine may simulate sintering using a finite element analysis (FEA) approach.

Some examples of the physics engine may utilize a time-marching approach. Starting at an initial time, the physics engine may simulate and/or process a simulation increment (e.g., a period of time, dt, etc.). In some examples, the simulation increment may be indicated by received input. For instance, the apparatus may receive an input from a user indicating the simulation increment. In some examples, the simulation increment may be selected randomly, may be selected from a range, and/or may be selected empirically.

In some examples, the physics engine may utilize trial displacements. A trial displacement is an estimate of a displacement that may occur during sintering. Trial displacements may be produced by a machine learning model and/or with another function (e.g., random selection and/or displacement estimating function, etc.). The trial displacements (e.g., trial displacement field) may trigger imbalances of the forces involved in sintering process. In some examples, the physics simulation engine may include and/or utilize an iterative optimization technique to iteratively re-shape displacements initialized by the trial displacements such that force equilibrium is achieved. In some examples, the physics simulation engine may produce a displacement field (e.g., equilibrium displacement field that may be denoted) as a sintering state at a simulation increment. The physics engine may produce a quantity of initial simulated sintering states by repeating operations for a quantity of simulation increments. The quantity of initial simulated sintering states may be limited. For instance, the physics engine may produce a quantity of initial simulated sintering states over a portion of the sintering procedure. Examples of the quantity of initial simulated sintering states may include 5, 10, 15, 50, 100, 500, etc., initial simulated sintering states.

In some examples, the apparatus may represent the initial sintering states as graphs. For instance, the apparatus may represent the voxels of an initial sintering state as nodes with attributes and as edges indicating interactions between voxels as described herein. Predicting 102 the first sintering state and predicting 104 the second sintering state may be based on the graphs.

In some examples, at the inferencing stage (after training, for instance), the first machine learning model, the second machine learning model, and the fusion machine learning model may take the quantity of initial simulated sintering state(s) (e.g., initial displacement vector(s) of initial increment(s), graph(s), etc.) as input, without further simulated sintering state(s). In some examples, the first machine learning model, the second machine learning model, and the fusion machine learning model may provide predictions until a last increment of the sintering procedure and output a final sintering state (e.g., final deformation vector) at a voxel level. In some examples, the complete inference run of the sintering procedure may indicate an additional checkpoint at an intermediate increment or transitioning stage without additional physics simulation.

In some approaches, prediction error may accumulate as the increments increase. The prediction error (e.g., deviation from a ground truth deformation value) may vary for different particles (e.g., voxels and/or nodes) in deviation scale and dimension. For example, the error for a first particle may be mainly on the x axis, while the error for a second particle may be mainly on the y axis. The nonuniformity of errors for different particles may change the correlation of displacements. Some examples of the techniques described herein may utilize an analysis of changes in displacement correlation (e.g., voxel displacement correlation) to suppress the accumulated error. For example, the fusion machine learning model may be trained based on a displacement correlation. For instance, the fusion machine learning model may be trained based on a loss function that includes the displacement correlation.

In some examples of the techniques described herein, k particles (e.g., voxels) may be randomly selected from an object surface. Displacement correlations may be calculated for each particle (e.g., each particle pair). An additional loss term may be utilized to align the correlation of predictions and that of the ground truth. In some examples, correlation coefficients may be defined in accordance with their respective cosine similarity in accordance with Equation (1).

$${C}_{ij}^{G} = \frac{{a}_{i} \cdot {a}_{j}}{‖ {a}_{i} ‖ ‖ {a}_{j} ‖} {C}_{ij}^{P} = \frac{{\hat{a}}_{i} \cdot {\hat{a}}_{j}}{‖ {\hat{a}}_{i} ‖ ‖ {\hat{a}}_{j} ‖} i , j ∈ \{ 1 , 2 , ... , k \} \qquad ( 1 )$$

In Equation (1), â is a predicted sintering state (e.g., predicted displacement vector, velocity, etc.), a is a ground truth sintering state (e.g., simulated sintering state, simulated displacement vector, velocity, etc.), k is an index of a randomly selected particle, Cᵢⱼ^{G} is a computed correlation among ground truth data (e.g., metal voxels' displacement vectors, sintering states, velocities, etc., in ground truth data), and Cᵢⱼ^{P} is a computed correlation among predicted data (e.g., metal voxels' displacement vectors, sintering states, velocities, etc., in predicted data).

In some examples, a correlation loss (L_{Corr}) may be expressed in accordance with Equation (2).

$${L}_{Corr} = \frac{1}{{k}^{2}} {\sum }_{i = 1}^{k} {\sum }_{j = 1}^{k} {( {C}_{ij}^{G} - {C}_{ij}^{P} )}^{2} \qquad ( 2 )$$

In some examples, a loss function to train the fusion machine learning model (e.g., RNN) may be expressed in accordance with Equation (3).

$$L = α {\sum }_{i = 1}^{n} {( {\hat{a}}_{i} - {a}_{i} )}^{2} + β {L}_{Corr} \qquad ( 3 )$$

In Equation (3), L is a loss, α is a hyperparameter and β is a hyperparameter.

In some examples, an element or elements of the method 100 may recur, may be repeated, and/or may be iterated. For instance, the apparatus may predict a subsequent sintering state or states. An iteration is an instance of a repetitive procedure or loop. For example, an iteration may include a sequence of operations that may iterate and/or recur. For instance, an iteration may be a series of executed instructions in a loop.

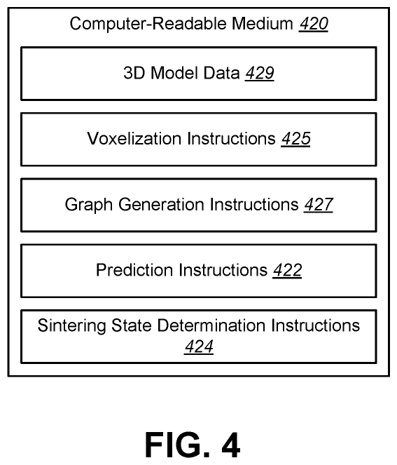

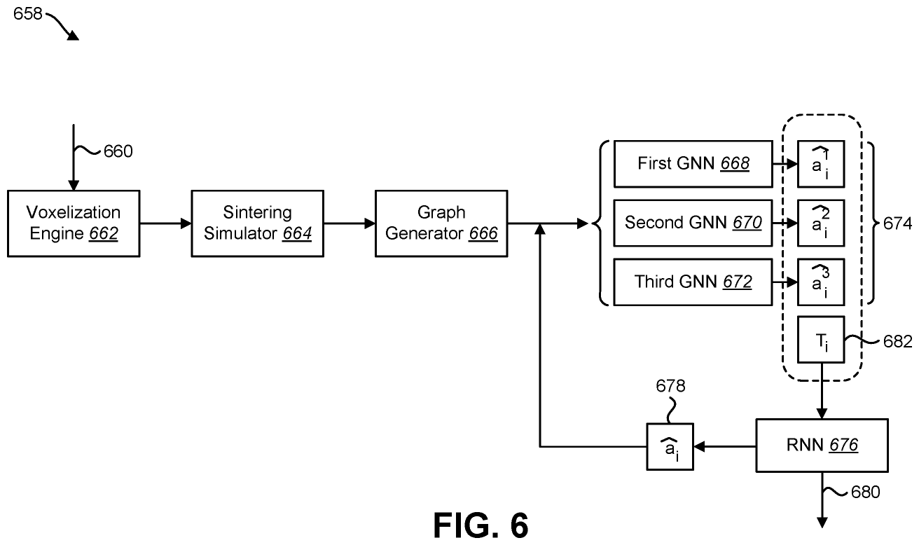

In some examples, operation(s), function(s), and/or element(s) of the method 100 may be omitted and/or combined. In some examples, the method 100 may include one, some, or all of the operation(s), function(s), and/or element(s) described in relation to FIG. 2, FIG. 3, FIG. 4, FIG. 5, and/or FIG. 6.

FIG. 2 is a diagram illustrating an example of a plot 201 of displacement and temperature in accordance with some of the techniques described herein. For example, the plot 201 illustrates examples of an x-axis displacement 217 and a y-axis displacement 219 corresponding to displacement at a point of maximum deformation in a shape deformation simulation. The x-axis displacement 217 and y-axis displacement 219 are illustrated in displacement in millimeters (mm) 209 over time in minutes 211 (in simulated time, for instance). Sintering procedure temperature 215 is illustrated in temperature in ° C. 213 over time in minutes 211 (in simulated time, for instance). While FIG. 2 illustrates examples of x-axis displacement 217, y-axis displacement 219, and sintering procedure temperature 215, some examples in accordance with the techniques described herein may include different sintering procedure temperature trends and/or different displacement. For instance, applied temperatures and/or sintering stages may vary.

As illustrated in the plot 201, a sintering procedure may include applying varying temperatures to cause an object to sinter. The object may experience deformation during the sintering procedure.

Examples of sintering stages are illustrated in FIG. 2. For example, the sintering procedure may include a first sintering stage 203, a second sintering stage 205, and a third sintering stage 207 (e.g., equilibrium stage). The first sintering stage 203 may have an associated time and/or temperature (e.g., 470-600 minutes), the second sintering stage 205 may have an associated time and/or temperature (e.g., 600-785 minutes), and the third sintering stage 207 may have an associated time and/or temperature (e.g., 785-900 minutes). In some examples, more or fewer sintering stages may be utilized. For instance, more or fewer sintering stages may be utilized for variations in sintering procedures (e.g., different heating patterns, different thermal profiles, different user settings, different sintering ovens, etc.). For instance, different sintering stages may be utilized for multiple temperature raises, different temperature raise rates, constant temperature periods, etc. In some examples, a respective machine learning model may be trained for each of the sintering stages. For example, a first machine learning model may be trained for the first sintering stage 203, a second machine learning model may be trained for the second sintering stage 205, and a third machine learning model may be trained for the third sintering stage 207. For instance, the simulation procedure may be partitioned into three sintering stages, where each sintering stage may have a respective machine learning model (e.g., deep learning model) based on sintering temperature profile, object geometry, and/or material. In some examples, the first machine learning model, the second machine learning model, and the third machine learning model may be GNNs.

In some examples, the first machine learning model, the second machine learning model, and the third machine learning models may be trained separately with data from respective time segments. For example, the first machine learning model may be trained with sintering deformation data in a first time segment 221 to learn the sintering deformation at the deformation rate of the first time segment 221. The prediction of the first machine learning model may be denoted at [symbol] . The second machine learning model may be trained with sintering deformation data from a second time segment 223. The second time segment 223 may correspond to the second sintering stage 205. The third machine learning model may be trained with sintering deformation data from a third time segment 225. The third time segment 225 may correspond to the third sintering stage 207. The predictions of the second machine learning model and the third machine learning model may be denoted [symbol] and [symbol] , respectively.

After training each machine learning model for the different sintering stages, a fusion machine learning model (e.g., RNN) may be trained over the sintering procedure in some examples. When training the fusion machine learning model, the trained machine learning models (e.g., GNNs) may be utilized to produce respective predictions for each increment. The predictions (e.g., [symbol] may be fed with temperature data to the fusion machine learning model (e.g., RNN) for fusion. The output of the fusion machine learning model may be the overall prediction (e.g., overall sintering state) for the corresponding increment. The overall prediction may be compared to the ground truth (ground truth deformation vectors at increment, for instance) for backward propagation. FIG. 5 illustrates an example of combining (e.g., fusing) sintering states using a fusion machine learning model (e.g., RNN) to produce an overall sintering state.

FIG. 3 is a block diagram of an example of an apparatus 302 that may be used in combining sintering states. The apparatus 302 may be a computing device, such as a personal computer, a server computer, a printer, a 3D printer, a smartphone, a tablet computer, etc. The apparatus 302 may include and/or may be coupled to a processor 304 and/or a memory 306. The memory 306 may be in electronic communication with the processor 304. For instance, the processor 304 may write to and/or read from the memory 306. In some examples, the apparatus 302 may be in communication with (e.g., coupled to, have a communication link with) an additive manufacturing device (e.g., a 3D printing device). In some examples, the apparatus 302 may be an example of a 3D printing device. The apparatus 302 may include additional components (not shown) and/or some of the components described herein may be removed and/or modified without departing from the scope of this disclosure.

The processor 304 may be any of a central processing unit (CPU), a semiconductor-based microprocessor, graphics processing unit (GPU), field-programmable gate array (FPGA), an application-specific integrated circuit (ASIC), and/or other hardware device suitable for retrieval and execution of instructions stored in the memory 306. The processor 304 may fetch, decode, and/or execute instructions (e.g., prediction instructions 312) stored in the memory 306. In some examples, the processor 304 may include an electronic circuit or circuits that include electronic components for performing a functionality or functionalities of the instructions (e.g., prediction instructions 312). In some examples, the processor 304 may perform one, some, or all of the functions, operations, elements, methods, etc., described in relation to one, some, or all of FIGS. 1-6.

The memory 306 may be any electronic, magnetic, optical, and/or other physical storage device that contains or stores electronic information (e.g., instructions and/or data). Thus, the memory 306 may be, for example, Random Access Memory (RAM), Electrically Erasable Programmable Read-Only Memory (EEPROM), a storage device, an optical disc, and/or the like. In some implementations, the memory 306 may be a non-transitory tangible machine-readable storage medium, where the term “non-transitory” does not encompass transitory propagating signals.

In some examples, the apparatus 302 may also include a data store (not shown) on which the processor 304 may store information. The data store may be volatile and/or non-volatile memory, such as Dynamic Random Access Memory (DRAM), EEPROM, magnetoresistive random-access memory (MRAM), phase change RAM (PCRAM), memristor, flash memory, and the like. In some examples, the memory 306 may be included in the data store. In some examples, the memory 306 may be separate from the data store. In some approaches, the data store may store similar instructions and/or data as that stored by the memory 306. For example, the data store may be non-volatile memory and the memory 306 may be volatile memory.

In some examples, the apparatus 302 may include an input/output interface (not shown) through which the processor 304 may communicate with an external device or devices (not shown), for instance, to receive and store the information pertaining to the object(s) for which a sintering state or states may be determined. The input/output interface may include hardware and/or machine-readable instructions to enable the processor 304 to communicate with the external device or devices. The input/output interface may enable a wired or wireless connection to the external device or devices. In some examples, the input/output interface may further include a network interface card and/or may also include hardware and/or machine-readable instructions to enable the processor 304 to communicate with various input and/or output devices, such as a keyboard, a mouse, a display, another apparatus, electronic device, computing device, etc., through which a user may input instructions into the apparatus 302. In some examples, the apparatus 302 may receive 3D model data 308 from an external device or devices (e.g., computer, removable storage, network device, etc.).

In some examples, the memory 306 may store 3D model data 308. The 3D model data 308 may be generated by the apparatus 302 and/or received from another device. Some examples of 3D model data 308 include a 3D manufacturing format (3MF) file or files, a 3D computer-aided design (CAD) image, object shape data, mesh data, geometry data, etc. The 3D model data 308 may indicate the shape of an object or objects. In some examples, the 3D model data 308 may indicate a packing of a build volume, or the apparatus 302 may arrange 3D object models represented by the 3D model data 308 into a packing of a build volume. In some examples, the 3D model data 308 may be utilized to obtain slices of a 3D model or models. For example, the apparatus 302 may slice the model or models to produce slices, which may be stored in the memory 306. In some examples, the 3D model data 308 may be utilized to obtain an agent map or agent maps of a 3D model or models. For example, the apparatus 302 may utilize the slices to determine agent maps (e.g., voxels or pixels where agent(s) are to be applied), which may be stored in the memory 306.

In some examples, the memory 306 may store graph generation instructions 314. The processor 304 may execute the graph generation instructions 314 to generate a graph representation based on the 3D model data 308. For instance, the processor 304 may voxelize an object model to produce voxels of the object model and may generate a graph representation of the voxels. In some examples, the graph representation of the object includes nodes corresponding to voxels of the object. The graph representation may be stored in the memory 306 as graph data 310. In some examples, generating the graph representation may be performed as described in relation to FIG. 1.

The memory 306 may store prediction instructions 312. In some examples, the processor 304 may execute the prediction instructions 312 to predict, using a first machine learning model, a first sintering state of an object based on a graph representation of the object. In some examples, this may be accomplished as described in relation to FIG. 1 and/or FIG. 2. For instance, the processor 304 may infer the sintering state (e.g., displacement, displacement rate of change, velocity, etc.) for an object represented by the graph data 310. In some examples, the first machine learning model may be trained using training data from a first segment.

In some examples, the processor 304 may execute the prediction instructions 312 to predict, using a second machine learning model, a second sintering state of the object based on the graph representation of the object. In some examples, this may be accomplished as described in relation to FIG. 1 and/or FIG. 2. For instance, the processor 304 may infer the sintering state (e.g., displacement, displacement rate of change, velocity, etc.) for an object represented by the graph data 310. In some examples, the second machine learning model may be trained using training data from a second segment. The first sintering state and the second sintering state may correspond to a same increment. In some examples, the first machine learning model and the second machine learning model may be GNNs.

In some examples, the processor 304 may execute the prediction instructions 312 to predict a deformation of the object based on the first sintering state, the second sintering state, and a temperature. In some examples, this may be accomplished as described in relation to FIG. 1 and/or FIG. 2. For example, the processor 304 may predict the deformation of the object using an RNN.

The memory 306 may store operation instructions 318. In some examples, the processor 304 may execute the operation instructions 318 to perform an operation based on the deformation. For example, the apparatus 302 may present the deformation and/or a value or values associated with the deformation (e.g., sintering state, maximum displacement, displacement direction, an image of the object model with a color coding showing the degree of displacement over the object model, etc.) on a display, may store the deformation and/or associated data in memory 306, and/or may send the deformation and/or associated data to another device or devices. In some examples, the apparatus 302 may determine whether a deformation (e.g., last or final deformation) is within a tolerance (e.g., within a target amount of displacement). In some examples, the apparatus 302 may print a precursor object based on the object model if the deformation is within the tolerance. For example, the apparatus 302 may print the precursor object based on two-dimensional (2D) maps or slices of the object model indicating placement of binder agent (e.g., glue). In some examples, the apparatus 302 (e.g., processor 304) may determine compensation based on the deformation (e.g., series of sintering states and/or final sintering state). For instance, the apparatus 302 (e.g., processor 304) may adjust the object model to compensate for the deformation (e.g., sag). For example, the object model may be adjusted in an opposite direction or directions from the displacement(s) indicated by the deformation.

FIG. 4 is a block diagram illustrating an example of a computer-readable medium 420 for combining sintering states. The computer-readable medium 420 may be a non-transitory, tangible computer-readable medium 420. The computer-readable medium 420 may be, for example, RAM, EEPROM, a storage device, an optical disc, and the like. In some examples, the computer-readable medium 420 may be volatile and/or non-volatile memory, such as DRAM, EEPROM, MRAM, PCRAM, memristor, flash memory, and/or the like. In some implementations, the memory 306 described in relation to FIG. 3 may be an example of the computer-readable medium 420 described in relation to FIG. 4.

The computer-readable medium 420 may include data (e.g., information, instructions, and/or executable code, etc.). For example, the computer-readable medium 420 may include 3D model data 429, voxelization instructions 425, graph generation instructions 427, prediction instructions 422, and/or sintering state determination instructions 424.

In some examples, the computer-readable medium 420 may store 3D model data 429. Some examples of 3D model data 429 include a 3D CAD file, a 3D mesh, etc. The 3D model data 429 may indicate the shape of a 3D object or 3D objects (e.g., object model(s)).

In some examples, the voxelization instructions 425 are code to cause a processor to voxelize an object model to produce voxels. In some examples, voxelizing the object model may be performed as described in relation to FIG. 1, FIG. 2, and/or FIG. 3.

In some examples, the graph generation instructions 427 are code to cause a processor to generate a graph representation of the voxels. In some examples, generating the graph representation may be performed as described in relation to FIG. 1 and/or FIG. 3.

In some examples, the prediction instructions 422 are code to cause a processor to predict a first sintering state for a first time increment using a first machine learning model. In some examples, predicting the first sintering state may be performed as described in relation to FIG. 1, FIG. 2, and/or FIG. 3.

In some examples, the prediction instructions 422 are code to cause a processor to predict a second sintering state for the first time increment using a second machine learning model. In some examples, predicting the second sintering state may be performed as described in relation to FIG. 1, FIG. 2, and/or FIG. 3.

In some examples, the sintering state determination instructions 424 are code to cause a processor to determine an overall sintering state for the first time increment using a third machine learning model based on the first sintering state and the second sintering state. In some examples, determining the overall sintering state may be performed as described in relation to FIG. 1, FIG. 2, and/or FIG. 3. In some examples, the third machine learning model is trained based on a voxel displacement correlation. In some examples, the first time increment is during a transition between a first segment and a second segment.

FIG. 5 is a diagram illustrating an example of a machine learning model architecture 526 that may be utilized in accordance with some of the techniques described herein. In some examples, the method 100 may utilize the architecture 526 to predict sintering states and/or combine sintering states. In some examples, the apparatus 302 may utilize (e.g., the processor 304 may execute) the architecture 526 to predict sintering states and/or combine sintering states.

In some examples, the machine learning model architecture 526 may include GNNs 528 and an RNN 530. For instance, GNN¹ may be an example of the first machine learning model and GNN² may be an example of the second machine learning model described in FIG. 1. In the example of FIG. 5, the machine learning model architecture 526 includes GNN¹, GNN², and GNN³, which may be trained based on respective time segments from different sintering stages of a sintering procedure. The RNN 530 may be an example of the fusion machine learning model described in relation to FIG. 1.

In the example of FIG. 5, the GNNs 528 may predict respective sintering states [symbol] , where i denotes an increment. For an increment i=1, for instance, the GNNs 528 may predict sintering states [symbol] based on a graph G⁽⁰⁾ 532.

[ [symbol] , Tᵢ]^{T} is an input of the RNN 530 for increment i, where Tᵢ denotes the temperature on increment i, and âᵢ denotes the output (e.g., overall sintering state, final prediction for increment i) from the RNN 530. On each increment, âᵢ may be applied to the graph data to update the particle displacements. In some examples, the loss function for training the RNN 530 may be expressed in accordance with Equation (3) above or Equation (4) (without the correlation loss, for instance).

$$L = {\sum }_{i = 1}^{n} {( {\hat{a}}_{i} - {a}_{i} )}^{2} \qquad ( 4 )$$

In Equation (4), âᵢ is a predicted sintering state for increment i and aᵢ is a ground truth sintering state for increment i. As illustrated in FIG. 5, the RNN 530 may output a series of predicted overall sintering states 534 by combining predicted sintering states from the GNNs 528.

FIG. 6 is a block diagram illustrating an example of components 658 that may be utilized in accordance with some examples of the techniques described herein. In some examples, the components 658 may be utilized to perform the method 100 described in relation to FIG. 1 and/or may be included in the apparatus 302 described in relation to FIG. 3. The components 658 may include a voxelization engine 662, a sintering simulator 664, a graph generator 666, a first GNN 668, a second GNN 670, a third GNN 672, and an RNN 676.

In the example of FIG. 6, object model data 660 is provided to the voxelization engine 662. The voxelization engine 662 may voxelize the object model data 660 to produce voxels. The voxels may be provided to the sintering simulator 664.

The sintering simulator 664 may simulate sintering to produce a quantity of initial simulated sintering states (e.g., deformation values). For instance, the sintering simulator 664 may produce initial simulated sintering states for L initial increments. The initial simulated sintering states may be provided to the graph generator 666.

The graph generator 666 may generate a graph based on the initial simulated sintering states. The graph may be provided to the first GNN 668, the second GNN 670, and the third GNN 672. The first GNN 668 may predict a first sintering state aᵢ¹, a second sintering state aᵢ², and a third sintering state aᵢ³ based on the initial simulated sintering states represented by the graph. A temperature (e.g., Tᵢ) 682 for the increment i may be appended to the predicted sintering states 674. The predicted sintering states 674 and the temperature 682 may be provided to the RNN 676.

The RNN 676 may predict an overall sintering state (e.g., âᵢ) 678 based on the predicted sintering states 674 and the temperature 682. The overall sintering state 678 may be utilized to update the graph. For instance, the overall sintering state displacements and/or velocities may be utilized to adjust the nodes and/or edges of the graph. The first GNN 668, the second GNN 670, and the third GNN 672 may utilize the updated graph to predict sintering states 674 for a subsequent increment. In some examples, a series of overall sintering states 680 (e.g., rollout transient predictions) corresponding to a sintering procedure may be stored.

Some examples of the techniques described herein provide approaches to fuse multiple models of a graph network to provide a quantitative model that can learn a physical sintering process and deliver prediction with increased speed. Some examples of the techniques described herein may provide automatic selection, transitioning, and/or integration of multiple deep learning inferencing models with a time-marching simulation. Some examples of the techniques described herein may provide fusing of predictions from multiple inferencing models trained for particular physics emphases (e.g., sintering stages). Some examples of the techniques described herein may provide inferencing models that take the initial sintering states of a physical simulation as input, and output an overall sintering state (e.g., deformation at a metal voxel level). Some examples of the techniques described herein may compute changes in voxels' displacement correlation and/or may suppress accumulated error in rollout predictions using the computed surface voxel displacement correlation.

As used herein, the term “and/or” may mean an item or items. For example, the phrase “A, B, and/or C” may mean any of: A (without B and C), B (without A and C), C (without A and B), A and B (without C), B and C (without A), A and C (without B), or all of A, B, and C.

While various examples of techniques are described herein, the techniques are not limited to the examples. Variations of the examples described herein may be implemented within the scope of the disclosure. For example, operations, functions, aspects, or elements of the examples described herein may be omitted or combined.

1. A method, comprising:
predicting a first sintering state of an object using a first machine learning model trained based on a first time segment;
predicting a second sintering state of the object using a second machine learning model trained based on a second time segment; and
combining, using a fusion machine learning model, the first sintering state and the second sintering state to produce an overall sintering state.

2. The method of claim 1, wherein the first machine learning model is a first graph neural network (GNN), the second machine learning model is a second GNN, and the fusion machine learning model is a recurrent neural network (RNN).

3. The method of claim 1, wherein the fusion machine learning model is trained to learn a dynamic of a sintering procedure based on the first time segment and the second time segment.

4. The method of claim 1, wherein the first machine learning model, the second machine learning model, and the fusion machine learning model are to produce the overall sintering state based on a quantity of initial simulated sintering states.

5. The method of claim 1, wherein the fusion machine learning model is trained based on a displacement correlation.

6. The method of claim 5, wherein the fusion machine learning model is trained based on a loss function that includes the displacement correlation.

7. The method of claim 1, further comprising voxelizing the object to produce voxels of the object.

8. The method of claim 7, further comprising simulating sintering of the voxels to produce a quantity of initial simulated sintering states.

9. The method of claim 8, further comprising representing the initial simulated sintering states as graphs, wherein predicting the first sintering state is based on the graphs and predicting the second sintering state is based on the graphs.

10. An apparatus, comprising:
a memory;
a processor in electronic communication with the memory, wherein the processor is to:
predict, using a first machine learning model, a first sintering state of an object based on a graph representation of the object;
predict, using a second machine learning model, a second sintering state of the object based on the graph representation of the object; and
predict a deformation of the object based on the first sintering state, the second sintering state, and a temperature.

11. The apparatus of claim 10, wherein the processor is to predict the deformation of the object using a recurrent neural network (RNN).

12. The apparatus of claim 10, wherein the graph representation of the object includes nodes corresponding to voxels of the object.

13. A non-transitory tangible computer-readable medium storing executable code, comprising:
code to cause a processor to voxelize an object model to produce voxels;
code to cause the processor to generate a graph representation of the voxels;
code to cause the processor to predict a first sintering state for a first time increment using a first machine learning model;
code to cause the processor to predict a second sintering state for the first time increment using a second machine learning model; and
code to cause the processor to determine an overall sintering state for the first time increment using a third machine learning model based on the first sintering state and the second sintering state.

14. The non-transitory tangible computer-readable medium of claim 13, wherein the third machine learning model is trained based on a voxel displacement correlation.

15. The non-transitory tangible computer-readable medium of claim 13, wherein the first time increment is during a transition between a first segment and a second segment.
